SEQUENCE LISTING (8 sequences)

SEQ ID NO: 1 - protein, 11 aa, Artificial Sequence
  source: /note=“Description of Artificial Sequence Synthetic peptide”
  misc_feature (1)..(1): /note=“May or may not be present”
  VARIANT (1)..(2): /replace=“Lys”
  misc_feature (1)..(2): /note=“This region may encompass ′Lys′ or ′Phe-Ile′, wherein one position may be absent”
  VARIANT (3)..(3): /replace=“Nle” or “Lys” or “Asp”
  VARIANT (4)..(8): /replace=“Lys” or “any hydrophobic amino acid”
  VARIANT (9)..(11): /replace=“Met” or “Asp” or “ ”
  misc_feature (9)..(11): /note=“This region may encompass ′Met,′ ′Asp′ or ′Leu-Leu-Ile,′ wherein some positions may be absent”
  misc_feature (1)..(11): /note=“Variant residues given in the sequence have no preference with respect to those in the annotations for variant positions”
FIMDDDDDLLI

SEQ ID NO: 2 - protein, 26 aa, Artificial Sequence
  source: /note=“Description of Artificial Sequence Synthetic peptide”
  source: /note=“N-term Fluo”
  source: /note=“C-term NH2”
MIILIIGSTSRDHMVLHEYVNAAGIT

SEQ ID NO: 3 - protein, 26 aa, Artificial Sequence
  source: /note=“Description of Artificial Sequence Synthetic peptide”
  source: /note=“N-term Fluo”
  misc_feature (1)..(6): /note=“Cyclic”
  MOD_RES (14)..(14): Nle
  source: /note=“C-term Ado-NH2”
KIIIIDGSTSRDHXVLHEYVNAAGIT

SEQ ID NO: 4 - protein, 28 aa, Artificial Sequence
  source: /note=“Description of Artificial Sequence Synthetic peptide”
  source: /note=“N-term Fluo”
  misc_feature (1)..(8): /note=“Cyclic”
  MOD_RES (2)..(2): Nle
  MOD_RES (16)..(16): Nle
  source: /note=“C-term Ado-NH2”
KXIILIIDGSTSRDHXVLHEYVNAAGIT

SEQ ID NO: 5 - protein, 26 aa, Artificial Sequence
  source: /note=“Description of Artificial Sequence Synthetic peptide”
  source: /note=“N-term Fluo”
  MOD_RES (1)..(1): Nle
  MOD_RES (14)..(14): Nle
  source: /note=“C-term Ado-NH2”
XIILIIGSTSRDHXVLHEYVNAAGIT

SEQ ID NO: 6 - protein, 26 aa, Artificial Sequence
  source: /note=“Description of Artificial Sequence Synthetic peptide”
  source: /note=“N-term Fluo”
  MOD_RES (1)..(1): D-Met
  MOD_RES (3)..(3): D-Ile
  MOD_RES (14)..(14): Nle
  source: /note=“C-term Ado-NH2”
MIILIIGSTSRDHXVLHEYVNAAGIT

SEQ ID NO: 7 - protein, 23 aa, Artificial Sequence
  source: /note=“Description of Artificial Sequence Synthetic peptide”
  source: /note=“N-term Fluo”
  source: /note=“C-term NH2”
MIILIIMGVADLIKKFESISKEE

SEQ ID NO: 8 - protein, 31 aa, Artificial Sequence
  source: /note=“Description of Artificial Sequence Synthetic polypeptide”
  source: /note=“N-term Fluo”
  misc_feature (3)..(7): /note=“Cyclic”
  MOD_RES (19)..(19): Nle
  source: /note=“C-term Ado-NH2”
FIDIIIKILLIGSTSRDHXVLHEYVNAAGIT

(19) United States
(12) Patent Application Publication
(10) Pub. No.: US 2017/0369529 A1
(43) Pub. Date: Dec. 28, 2017

(54) CELL PENETRATING PEPTIDES

(71) Applicant: Hoffmann-La Roche Inc., Little Falls, NJ (US)

(72) Inventor: Francesca Milletti, New York, NY (US)

(21) Appl. No.: 15/529,649

(22) PCT Filed: Dec. 18, 2015
(86) PCT No.: PCT/EP2015/080385

§ 371 Date: May 25, 2017

Related U.S. Application Data
(60) Provisional application No. 62/095,198, filed on Dec. 22, 2014.

(51) Int. Cl. C07K 7/06; A61K 38/00; C07K 14/435; C07K 19/00; A61K 47/64; A61K 49/00

(52) U.S. Cl. CPC C07K 7/06; A61K 38/00; C07K 14/435; A61K 47/64; A61K 49/0043; C07K 19/00; C07K 2319/10; A61K 49/0056

(57) ABSTRACT

Provided herein are cell penetrating peptides optionally including a cargo moiety linked thereto.

### SEQUENCE LISTING

The instant application contains a Sequence Listing which has been submitted electronically in ASCII format and is hereby incorporated by reference in its entirety. Said ASCII copy, created on Nov. 2, 2015, is named 113123-89625_SL.txt and is 5,622 bytes in size.

### FIELD OF THE INVENTION

The present invention relates to peptides that can penetrate a cell and optionally carry cargo molecules into the cell. All documents cited to or relied upon below are expressly incorporated herein by reference.

### BACKGROUND OF THE INVENTION

A large number of attractive drug targets are intracellular protein-protein interactions (PPIs). However, PPIs cannot be easily modulated by traditional molecules, which are too small, and are inaccessible to larger compounds such as peptides, which typically cannot cross the cell membrane.

Cell-penetrating peptides (CPPs) are a class of diverse peptides, typically with 5-30 amino acids, that unlike most peptides can cross the cellular membrane. Since the discovery of the first CPP, penetratin (Derossi et al, Biol. Chem., 269, 10444-10450, 1994), CPPs have been used for a variety of applications. CPPs can act as vectors for nucleic acids (Lehto et al. Exp. Op. Drug Delivery, 9, 823-836, 2012), small molecules, proteins, and for other peptides, both in vitro and in vivo (Copolovici et al, ACS Nano, 8, 1972-1994, 2014). Not only can a CPP be used to carry a functional peptide inside the cell, but it can also incorporate a functional motif.

Initially, cellular uptake was believed to occur by direct permeation of the plasma membrane (Prochiantz, Curr. Opin. Cell Biol, 12, 400-406, 2000) but we now know that endocytosis contributes significantly to the cellular uptake (Fotin-Mleczek et al, Curr. Pharm. Design, 11, 3613-3628, 2005). Given these recent results, the specification of a peptide as a CPP does not imply a specific cellular import mechanism, but rather refers to the ability of a peptide to enhance the cellular uptake of the cargo molecule to which it is covalently or noncovalently conjugated.

A need exists in the art for novel CPPs for administering a peptide or a peptide-cargo conjugate to a patient or subject in need thereof.

### SUMMARY OF THE INVENTION

The invention relates to a peptide, comprising the sequence of SEQ ID No. 1:

X_{A}-X_{B}-X_{C}-X_{D}-X_{E}-X_{F}-X_{G}-X_{H} (SEQ ID No. 1),

wherein:
X_{A} is absent or is Lys or Phe-Ile;

### X_{B} is Met, Norleucine, Lys or Asp;

X_{C}, X_{D}, X_{E}, X_{F} and X_{G} are, independently of each other, a hydrophobic amino acid, Asp or Lys; and
X_{H} is absent or is Met, Asp or Leu-Leu-Ile,
said peptide optionally comprising a cargo moiety linked to a position on said sequence of SEQ ID No. 1 to form a peptide-cargo conjugate.

The invention further relates to a pharmaceutical composition, comprising a therapeutically effective amount of a peptide-cargo conjugate.

The invention additionally relates to a method for the treatment of cancer or a virological, central nervous system, inflammatory, immune, or metabolic disease or condition, comprising the step of administering a therapeutically effective amount of a peptide-cargo conjugate to a patient in need thereof.

The invention also relates to an isolated nucleotide encoding a peptide and to a vector comprising said isolated nucleotide.

### DETAILED DESCRIPTION OF THE INVENTION

It is to be understood that the figures and descriptions of the present invention have been simplified to illustrate elements that are relevant for a clear understanding of the present invention, while eliminating, for the purpose of clarity, many other elements found in typical peptide synthesis. Those of ordinary skill in the art will recognize that other elements and/or steps are desirable and/or required in implementing the present invention. However, because such elements and steps are well known in the art, and because they do not facilitate a better understanding of the present invention, a discussion of such elements and steps is not provided herein. The disclosure herein is directed to all such variations and modifications to such elements and methods known to those skilled in the art. Furthermore, the embodiments identified and illustrated herein are for exemplary purposes only, and are not meant to be exclusive or limited in their description of the present invention.

All peptide sequences mentioned herein are written according to the usual convention whereby the N-terminal amino acid is on the left and the C-terminal amino acid is on the right. A short line between two amino acid residues indicates a peptide bond. Where the amino acid has isomeric forms, it is the L form of the amino acid that is represented unless otherwise expressly indicated.

For convenience in describing this invention, the conventional and nonconventional abbreviations for the various amino acids residues are used. These abbreviations are familiar to those skilled in the art, but for clarity are listed below:

Asp=D=Aspartic Acid; Ala=A=Alanine; Arg=R=Arginine; Asn=N=Asparagine; Gly=G=Glycine; Glu=E=Glutamic Acid; Gln=Q=Glutamine; His=H=Histidine; Ile=I=Isoleucine; Leu=L=Leucine; Lys=K=Lysine; Met=M=Methionine; Phe=F=Phenylalanine; Pro=P=Proline; Ser=S=Serine; Thr=T=Threonine; Trp=W=Tryptophan; Tyr=Y=Tyrosine; and Val=V=Valine; Nle=Norleucine; Fluo=carboxyfluorescein; Ado=9-(Fmoc-amino)-3,6-dioxa-octanoic acid.

Amino acids can be in either L- or D-form. D-amino acids are referred to in the lower case, L-amino acids are referred to in the uppercase.

Also for convenience, and readily known to one skilled in the art, the following abbreviations or symbols are used to represent the moieties, reagents and the like used herein:

Et₂O is diethyl ether; hr(s) is hour(s); TIS is triisopropylsilane; ACN is acetonitrile, Fmoc is 9-fluorenylmethyloxycarbonyl; DMF is dimethylformamide; DIPEA is N,N-diisopropyl-ethylamine; TFA is trifluoroacetic acid; HOBT is N-hydroxybenzotriazole; BOP is benzo-triazol-1-yloxy-tris-(dimethylamino)phosphonium-hexafluorophosphate; HBTU is 2-(1H-benzotriazole-1-yl)-1,1,3,3-tetramethyluronium-hexafluorophosphate; (ES)+-LCMS is electro spray liquid chromatography-mass spectrometry; DIEA is diisopropylethylamine; MeOH is methanol; and DCM is methylene chloride.
Hydrophobic amino acids useful in the present invention include Leu, Ile, Phe, Trp, Val, Met, Cys, Tyr and Ala.

Thus, in one embodiment of the present invention, provided is a peptide, comprising the sequence of SEQ ID No. 1:

X_{A}-X_{B}-X_{C}-X_{D}-X_{E}-X_{F}-X_{G}-X_{H} (SEQ ID No. 1),

wherein:
X_{A} is absent or is Lys or Phe-Ile;

### X_{B} is Met, Norleucine, Lys or Asp;

X_{C}, X_{D}, X_{E}, X_{F} and X_{G} are, independently of each other, a hydrophobic amino acid, Asp or Lys; and
X_{H} is absent or is Met, Asp or Leu-Leu-Ile,
said peptide optionally comprising a cargo moiety linked to a position on said sequence of SEQ ID No. 1 to thereby form a peptide-cargo conjugate.

In another embodiment of the invention, provided is a peptide, comprising the sequence of SEQ ID No. 1, wherein the hydrophobic amino acid is selected from the group consisting of Leu, Ile, Phe, Trp, Val, Met, Cys, Tyr and Ala.

In another embodiment of the invention, provided is a peptide, comprising the sequence of SEQ ID No. 1, wherein said cargo moiety is a peptide, polypeptide, protein, small molecular substance, drug, mononucleotide, oligonucleotide, polynucleotide, antisense molecule, double stranded as well as single stranded DNA, RNA, an artificial or partly artificial nucleic acid, a low molecular weight molecule, saccharide, plasmid, antibiotic substance, cytotoxic agent, antiviral agent or a tag or marker molecule.

In another embodiment of the invention, provided is a peptide, comprising the sequence of SEQ ID No. 1, wherein said peptide further comprises a lactam, thioether or disulfide bridge.

In another embodiment of the invention, provided is a peptide, comprising the sequence of SEQ ID No. 1, wherein said cargo moiety is linked to X_{H} or, in the absence of X_{H}, to X_{G}.

In another embodiment of the invention, provided is a peptide, comprising the sequence of SEQ ID No. 1, wherein said cargo moiety is linked to X_{A} or, in the absence of X_{A}, to X_{B}.

In another embodiment of the invention, provided is a peptide, comprising the sequence of SEQ ID No. 1, wherein X_{A} is absent.

In another embodiment of the invention, provided is a peptide, comprising the sequence of SEQ ID No. 1, wherein X_{A} is Lys or Phe-Ile.

In another embodiment of the invention, provided is a peptide, comprising the sequence of SEQ ID No. 1, wherein X_{B} is Met or Norleucine.

In another embodiment of the invention, provided is a peptide, comprising the sequence of SEQ ID No. 1, wherein X_{B} is Lys or Asp.

In another embodiment of the invention, provided is a peptide, comprising the sequence of SEQ ID No. 1, wherein X_{C}, X_{D} and X_{E} independently are Be or Leu.

In another embodiment of the invention, provided is a peptide, comprising the sequence of SEQ ID No. 1, wherein X_{F} and X_{G} independently are Ile or Leu.

In another embodiment of the invention, provided is a peptide, comprising the sequence of SEQ ID No. 1, wherein X_{H} is absent.

In another embodiment of the invention, provided is a peptide, comprising the sequence of SEQ ID No. 1, wherein X_{H} is Met, Asp or Leu-Leu-Ile.

In another embodiment of the invention, provided is a peptide, comprising the sequence of SEQ ID No. 1, wherein X_{H} is Met or Leu-Leu-Ile.

In another embodiment of the invention, provided is a peptide, comprising the sequence of SEQ ID No. 1, wherein X_{H} is Asp.

In another embodiment of the invention, provided is a peptide, comprising the sequence of SEQ ID No. 1, wherein X_{A} and X_{H} are linked to form a lactam bridge.

In another embodiment of the invention, provided is a peptide, comprising the sequence of SEQ ID No. 1, wherein X_{B} and X_{G} are linked to form a lactam bridge.

In another embodiment of the invention, provided is a peptide, comprising the sequence of SEQ ID No. 1, wherein X_{B} and X_{F} are linked to form a lactam bridge.

In another embodiment of the invention, provided is a peptide, comprising the sequence of SEQ ID No. 1, wherein said cargo moiety is linked to X_{H}.

In another embodiment of the invention, provided is a peptide, comprising the sequence of SEQ ID No. 1, wherein X_{H} is absent and said cargo moiety is linked to X_{G}.

In another embodiment of the invention, provided is a peptide, comprising the sequence of SEQ ID No. 1, wherein:

X_{A} is absent;

### X_{B} is Met or Norleucine;

X_{C}, X_{D} and X_{E} independently are a hydrophobic amino acid;
X_{F} and X_{G} independently are a hydrophobic amino acid;
X_{H} is absent or is Met;
and wherein X_{H} or, in the absence of X_{H}, X_{G} is optionally linked to said cargo moiety.

In another embodiment of the invention, provided is a peptide, comprising the sequence of SEQ ID No. 1, wherein:

X_{A} is absent;

### X_{B} is Met or Norleucine;

X_{C}, X_{D} and X_{E} independently are Ile or Leu;
X_{F} and X_{G} independently are a hydrophobic amino acid;
X_{H} is absent or is Met;
and wherein X_{H} or, in the absence of X_{H}, X_{G} is optionally linked to said cargo moiety.

In another embodiment of the invention, provided is a peptide, comprising the sequence of SEQ ID No. 1, wherein:

X_{A} is absent;

### X_{B} is Met or Norleucine;

X_{C}, X_{D} and X_{E} independently are Ile or Leu;
X_{F} and X_{G} are Ile;
X_{H} is absent or is Met;
and wherein X_{H} or, in the absence of X_{H}, X_{G} is optionally linked to said cargo moiety.

In another embodiment of the invention, provided is a peptide, comprising the sequence of SEQ ID No. 1, wherein:

X_{A} is absent;

### X_{B} is Met or Norleucine;

X_{C}, X_{D} and X_{E} independently are Ile or Leu;
X_{F} and X_{G} are Ile;
X_{H} is absent;
and wherein X_{G} is optionally linked to said cargo moiety.

In another embodiment of the invention, provided is a peptide, comprising the sequence of SEQ ID No. 1, wherein:

X_{A} is absent or is Lys or Phe-Ile;

### X_{B} is Norleucine, Lys or Asp;

X_{C}, X_{D} and X_{E} independently are a hydrophobic amino acid;
X_{F} and X_{G} independently are a hydrophobic amino acid;
X_{H} is absent or is Met, Asp or Leu-Leu-Ile;
X_{A} or X_{B} and X_{F}, X_{G} or X_{H} are optionally linked to form a lactam bridge; and wherein X_{H} or, in the absence of X_{H}, X_{G} is optionally linked to said cargo moiety.

In another embodiment of the invention, provided is a peptide, comprising the sequence of SEQ ID No. 1, wherein:

X_{A} is absent or is Lys or Phe-Ile;

### X_{B} is Norleucine, Lys or Asp;

X_{C}, X_{D} and X_{E} independently are Ile, Leu or Val;
X_{F} and X_{G} independently are a hydrophobic amino acid selected from Ile, Leu or Val, or Asp or Lys;
X_{H} is absent or is Met, Asp or Leu-Leu-Ile;
one of X_{A} or X_{B} and one of X_{F}, X_{G} or X_{H} are optionally linked to form a lactam bridge; and wherein X_{H} or, in the absence of X_{H}, X_{G} is optionally linked to said cargo moiety.

In another embodiment of the invention, provided is a peptide, comprising the sequence of SEQ ID No. 1, wherein:

X_{A} is absent or is Lys or Phe-Ile;

### X_{B} is Norleucine, Lys or Asp;

X_{C}, X_{D} and X_{E} independently are Ile, Leu or Val;
X_{F} and X_{G} independently are Ile, Leu or Val, or Asp or Lys;
X_{H} is absent or is Met, Asp or Leu-Leu-Ile;
X_{A} or X_{B} and X_{F}, X_{G} or X_{H} are optionally linked to form a lactam bridge;
and wherein X_{H} or, in the absence of X_{H}, X_{G} is optionally linked to said cargo moiety.

In another embodiment of the invention, provided is a peptide, comprising the sequence of SEQ ID No. 1, wherein:

X_{A} is absent or is Lys or Phe-Ile;

### X_{B} is Norleucine, Lys or Asp;

X_{C}, X_{D} and X_{E} independently are Ile or Leu;
X_{F} and X_{G} independently are Ile, Leu or Val, or Asp or Lys;
X_{H} is absent or is Met, Asp or Leu-Leu-Ile;
X_{A} or X_{B} and X_{F}, X_{G} or X_{H} are optionally linked to form a lactam bridge;
and wherein X_{H} or, in the absence of X_{H}, X_{G} is optionally linked to said cargo moiety.

In another embodiment of the invention, provided is a peptide, comprising the sequence of SEQ ID No. 1, wherein:

X_{A} is absent or is Lys or Phe-Ile;

### X_{B} is Norleucine, Lys or Asp;

X_{C}, X_{D} and X_{E} independently are Ile or Leu;
X_{F} and X_{G} independently are Ile, Asp or Lys;
X_{H} is absent or is Met, Asp or Leu-Leu-Ile;
X_{A} or X_{B} and X_{F}, X_{G} or X_{H} are optionally linked to form a lactam bridge; and wherein X_{H} or, in the absence of X_{H}, X_{G} is optionally linked to said cargo moiety.

In another embodiment of the invention, provided is a peptide, comprising the sequence of SEQ ID No. 1, wherein:

X_{A} is absent or is Lys or Phe-Ile;

### X_{B} is Norleucine, Lys or Asp;

X_{C}, X_{D} and X_{E} independently are Ile or Leu;
X_{F} and X_{G} independently are Ile, Asp or Lys;
X_{H} is absent or is Met, Asp or Leu-Leu-Ile;
X_{A} or X_{B} and X_{F}, X_{G} or X_{H} are optionally linked to form a lactam bridge;
and wherein X_{H} or, in the absence of X_{H}, X_{G} is optionally linked to said cargo moiety.

In a further embodiment of the present invention, provided is a peptide-cargo conjugate, comprising:

a peptide comprising the sequence of SEQ ID No. 1:

X_{A}-X_{B}-X_{C}-X_{D}-X_{E}-X_{F}-X_{G}-X_{H} (SEQ ID No. 1),

wherein:
X_{A} is absent or is Lys or Phe-Ile;

### X_{B} is Met, Norleucine, Lys or Asp;

X_{C}, X_{D}, X_{E}, X_{F} and X_{G} are, independently of each other, a hydrophobic amino acid, Asp or Lys; and
X_{H} is absent or is Met, Asp or Leu-Leu-Ile; and
a cargo moiety linked to a position on said sequence of SEQ ID No. 1.

In a still further embodiment of the present invention, provided is a peptide-cargo conjugate comprising a peptide comprising the sequence of SEQ ID NO 1 and a cargo moiety, wherein the cargo moiety is a peptide, polypeptide, protein, small molecular substance, drug, mononucleotide, oligonucleotide, polynucleotide, antisense molecule, double stranded as well as single stranded DNA, RNA, an artificial or partly artificial nucleic acid, a low molecular weight molecule, saccharide, plasmid, antibiotic substance, cytotoxic agent, antiviral agent or a tag or marker molecule.

In another embodiment of the invention, provided is a pharmaceutical composition, comprising a therapeutically effective amount of the peptide-cargo conjugate according to an embodiment above.

In another embodiment of the invention, provided is a method for the treatment of a cancerous, infectious, neurological, inflammatory, immunological, ocular or metabolic disease or disorder, comprising the step of administering a therapeutically effective amount of a peptide-cargo conjugate according to an embodiment the invention above to a patient in need thereof.

### General Synthesis of Certain Embodiments of the Invention

In general, the peptides of the present invention may be readily synthesized by any known conventional procedure for the formation of a peptide linkage between amino acids.

Such conventional procedures for synthesizing the peptides of the present invention include, e.g., any solid phase peptide synthesis method. In such a method the synthesis of the peptides can be carried out by sequentially incorporating the desired amino acid residues one at a time into the growing peptide chain according to the general principles of solid phase methods. Such methods are disclosed in, e.g., Merrifield, R. B., J. Amer. Chem. Soc. 85, 2149-2154 (1963); Barany et al, The Peptides, Analysis, Synthesis and Biology, Vol. 2, Gross, E. and Meienhofer, J., Eds. Academic Press 1-284 (1980), which are incorporated herein by reference.

In general, the peptides of the present invention may be readily synthesized by any known conventional procedure for the formation of a peptide linkage between amino acids. Such conventional procedures include, e.g., any solution phase procedure permitting a condensation between the free alpha amino group of an amino acid or fragment thereof having its carboxyl group and other reactive groups protected and the free primary carboxyl group of another amino acid or fragment thereof having its amino group or other reactive groups protected.

During the synthesis of peptides, it may be desired that certain reactive groups on the amino acid, e.g., the alpha-amino group, a hydroxyl group, and/or reactive side chain groups, be protected to prevent a chemical reaction therewith. This may be accomplished, e.g., by reacting the reactive group with a protecting group which may later be removed. For example, the alpha amino group of an amino acid or fragment thereof may be protected to prevent a chemical reaction therewith while the carboxyl group of that amino acid or fragment thereof reacts with another amino acid or fragment thereof to form a peptide bond. This may be followed by the selective removal of the alpha amino protecting group to allow a subsequent reaction to take place at that site, e.g. with the carboxyl group of another amino acid or fragment thereof.

Alpha amino groups may, e.g., be protected by a suitable protecting group selected from aromatic urethane-type protecting groups, such as allyloxycarbony, benzyloxycarbonyl (Z) and substituted benzyloxycarbonyl, such as p-chlorobenzyloxycarbonyl, p-nitrobenzyl-oxycarbonyl, p-bromobenzyloxycarbonyl, p-biphenyl-isopropyloxycarbonyl, 9-fluorenylmethyloxycarbonyl (Fmoc) and p-methoxybenzyloxycarbonyl (Moz); and aliphatic urethane-type protecting groups, such as t-butyloxycarbonyl (Boc), diisopropylmethyloxycarbonyl, isopropyloxycarbonyl, and allyloxycarbonyl. In an embodiment, Fmoc is used for alpha amino protection.

Hydroxyl groups (OH) of the amino acids may, e.g., be protected by a suitable protecting group selected from benzyl (Bzl), 2,6-dichlorobenzyl (2,6 diCl-Bzl), and tert-butyl (t-Bu). In an embodiment wherein a hydroxyl group of tyrosine, serine, or threonine is intended to be protected, t-Bu may, e.g., be used.

Epsilon-amino acid groups may, e.g., be protected by a suitable protecting group selected from 2-chloro-benzyloxycarbonyl (2-Cl—Z), 2-bromo-benzyloxycarbonyl (2-Br—Z), allycarbonyl and t-butyloxycarbonyl (Boc). In an embodiment wherein an epsilon-amino group of lysine is intended to be protected, Boc may, e.g., be used.

Beta- and gamma-amide groups may, e.g., be protected by a suitable protecting group selected from 4-methyltrityl (Mtt), 2, 4, 6-trimethoxybenzyl (Tmob), 4, 4′-dimethoxy-dityl (Dod), bis-(4-methoxyphenyl)-methyl and Trityl (Trt). In an embodiment wherein an amide group of asparagine or glutamine is intended to be protected, Trt may, e.g., be used.

Indole groups may, e.g., be protected by a suitable protecting group selected from formyl (For), Mesityl-2-sulfonyl (Mts) and t-butyloxycarbonyl (Boc). In an embodiment wherein the indole group of tryptophan is intended to be protected, Boc may, e.g., be used.

Imidazole groups may, e.g., be protected by a suitable protecting group selected from Benzyl (Bzl), t-butyloxycarbonyl (Boc), and Trityl (Trt). In an embodiment wherein the imidazole group of histidine is intended to be protected, Trt may, e.g., be used.

Solid phase synthesis may be commenced from the C-terminal end of the peptide by coupling a protected alpha-amino acid to a suitable resin. Such a starting material can be prepared by attaching an alpha-amino-protected amino acid by an ester linkage to a p-benzyl-oxybenzyl alcohol (Wang) resin, or by an amide bond between an Fmoc-Linker, such as a Rink linker, and a benzhydrylamine (BHA) resin. Preparation of the hydroxymethyl resin is well known in the art. Fmoc-Linker-BHA resin supports are commercially available and generally used when the desired peptide being synthesized has an unsubstituted amide at the C-terminus.

In an embodiment, peptide synthesis is microwave assisted. Microwave assisted peptide synthesis is an attractive method for accelerating the solid phase peptide synthesis. This may be performed using Microwave Peptide Synthesizer, e.g. a Liberty peptide synthesizer (CEM Corporation, Matthews, N.C.). Microwave assisted peptide synthesis allows for methods to be created that control a reaction at a set temperature for a set amount of time. The synthesizer automatically regulates the amount of power delivered to the reaction to keep the temperature at the set point.

Typically, the amino acids or mimetic are coupled onto the Fmoc-Linker-BHA resin using the Fmoc protected form of amino acid or mimetic, with 2-5 equivalents of amino acid and a suitable coupling reagent. After coupling, the resin may be washed and dried under vacuum. Loading of the amino acid onto the resin may be determined by amino acid analysis of an aliquot of Fmoc-amino acid resin or by determination of Fmoc groups by UV analysis. Any unreacted amino groups may be capped by reacting the resin with acetic anhydride and diispropylethylamine in methylene chloride.

The resins are carried through several repetitive cycles to add amino acids sequentially. The alpha amino Fmoc protecting groups are removed under basic conditions. Piperidine, piperazine or morpholine (20-40% v/v) in DMF may be used for this purpose. In an embodiment, 20% piperidine in DMF is utilized.

Following the removal of the alpha amino protecting group, the subsequent protected amino acids are coupled stepwise in the desired order to obtain an intermediate, protected peptide-resin. The activating reagents used for coupling of the amino acids in the solid phase synthesis of the peptides are well known in the art. For example, appropriate reagents for such syntheses are benzotriazol-1-yloxy-tri-(dimethylamino) phosphonium hexafluorophosphate (BOP), bromo-tris-pyrrolidino-phosphonium hexafluorophosphate (PyBroP) 2-(1H-benzotriazole-1-yl)-1,1,3,3-tetramethyluronium hexafluorophosphate (HBTU), and di-isopropylcarbodiimide (DIC). In an embodiment, the reagent is HBTU or DIC. Other activating agents are described by Barany and Merrifield (in The Peptides, Vol. 2, J. Meienhofer, ed., Academic Press, 1979, pp 1-284). Various reagents such as 1 hydroxybenzotriazole (HOBT), N-hydroxysuccinimide (HOSu) and 3,4-dihydro-3-hydroxy-4-oxo-1,2,3-benzo-triazine (HOOBT) may be added to the coupling mixtures in order to optimize the synthetic cycles. In an embodiment, HOBT is added.

Following synthesis of the peptide, the blocking groups may be removed and the peptide cleaved from the resin by any known method. For example, the peptide-resins may be treated with ethanedithiol, dimethylsulfide, anisole, and trifluoroacetic acid to remove the blocking group.

Purification of the crude peptide may be performed by any method known in the art. For example, purification can be performed on a Shimadzu LC-8A system by high performance liquid chromatography (HPLC) on a reverse phase C18 Column.

### Utility And Conjugation of the Peptides of the Present Invention

In a particular embodiments, the peptides of the present invention are “conjugated,” also referred to interchangeably herein as “linked” or “bound,” to one or more cargo moieties for delivery to the inside of cells (such as the cytoplasm or nucleus) for various therapeutic and other applications. Examples of such cargo moieties include, but are not limited to, the cargo disclosed in US2008/0234183 incorporated herein by reference in its entirety. For example, the cargo moiety may be any pharmacologically interesting substance, such as a peptide, polypeptide, protein, small molecular substance, drug, mononucleotide, oligonucleotide, polynucleotide, antisense molecule, double stranded as well as single stranded DNA, RNA and/or any artificial or partly artificial nucleic acid, e.g. PNA, a low molecular weight molecule, saccharide, plasmid, antibiotic substance, a cytotoxic agent or an antiviral agent or combinations thereof. Furthermore, the transport of cargo can be useful as a research tool for delivering e.g. tags and markers as well as for changing membrane potentials and/or properties, the cargo may e.g. be a marker molecule, such as biotin.

The cargo moiety/moieties can be conjugated to the peptide to form the peptide-cargo conjugate by known methods in the art. For example, the cargo may be conjugated to the peptide at any stage of peptide synthesis. In one embodiment, the cargo moiety can be conjugated after the peptide is fully synthesized. In another embodiment, the cargo moiety can added to a partially synthesized peptide.

The peptides and/or peptide-cargo conjugates of the invention are provided for use in the treatment of disease. The use of a peptide and/or a peptide-cargo conjugate in the manufacture of a medicament for the treatment of disease is also provided. A method of treatment of a patient or subject in need of treatment for a disease condition is also provided comprising the step of administering a therapeutically effective amount, as determined by a physician or veterinarian, of a peptide and/or a peptide-cargo conjugate to the patient or subject in need thereof. In one embodiment, the cargo component of a peptide-cargo conjugate comprises an active agent (e.g. pharmaceutical agent) capable of treating, preventing or ameliorating the disease.

Using CPPs for delivering conjugated cargo to the inside of cells and methods of conjugating or linking cargo such as small molecules, nucleic acids, fluorescent moieties, proteins, peptides and/or other cargo are well known in the art. See e.g. US2008/0234183; Rhee et al, 201. C105Y, a Novel Cell Penetrating Peptide Enhances Gene Transfer of Sec-R Targeted Molecular Conjugates, Molecular Therapy (2005) 11, S79-S79; Johnson et al, Cell-penetrating Peptide for Enhanced Delivery of Nucleic Acids and Drugs to Ocular Tissues Including Retina and Cornea, Molecular Therapy (2007) 16 (1), 107-114; El-Andaloussi et al, A Novel Cell-penetrating Peptide, M918, for Efficient Delivery of Proteins and Peptide Nucleic Acids, Molecular Therapy (2007) 15 (10), 1820-1826; and Crombez et al, A New Potent Secondary Amphipathic Cell-Penetrating Peptide for siRNA Delivery Into Mamma-lian Cells, Molecular Therapy (2008) 17 (1), 95-103; Sasaki, Y. et al, Cell-penetrating peptide-conjugated XIAP-inhibitory cyclic hexapeptides enter into Jurkat cells and inhibit cell proliferation FEBS Journal (2008) 275 (23), 6011-6021; Kolluri, S. K. et al, A Short Nur77-Derived Peptide Converts Bcl-2 from a Protector to a Killer, Cancer Cell (2008) 14 (4), 285-298; Avbelj, M., The Role of Intermediary Domain of MyD88 in Cell Activation and Therapeutic Inhibition of TLRs J. Immunology (2011), 1; 187(5):2394-404.

In addition, the foregoing examples demonstrate the conjugation to carboxyfluorescein and their subsequent cell penetration as summarized in the cell assay example below.

### EXAMPLES

The disclosure is further illustrated by the following examples, which are not to be construed as limiting this disclosure in scope or spirit to the specific procedures herein described. It is to be understood that the examples are provided to illustrate certain embodiments and that no limitation to the scope of the disclosure is intended thereby. It is to be further understood that resort may be had to various other embodiments, modifications, and equivalents thereof which may suggest themselves to those skilled in the art without departing from the spirit of the present disclosure and/or scope of the appended claims.

The peptides in the specific examples below were prepared by solid state synthesis. See Steward and Young, Solid Phase Peptide Synthesis, Freemantle, San Francisco, Calif. (1968). A preferred method is the Merrifield process. Merrifield, Recent Progress in Hormone Res., 23:451 (1967). In addition, the peptides in the specific examples below were synthesized by tagging the N-terminus of the peptide with FITC as a green fluorescent dye.

### Example 1

### Synthesis of Fluo-Met-Ile-Ile-Leu-Ile-Ile-Gly-Ser-Thr-Ser-Arg-Asp-His-Met-Val-Leu-His-Glu-Tyr-Val-Asn-Ala-Ala-Gly-Ile-Thr-NH₂ (SEQ ID NO: 2)

All chemicals and solvents such as DMF, DCM, DIEA and MeOH were used without further purification. Fmoc-Rink Amide MBHA resin (0.23 g, 0.1 mmol) was swollen in DMF, followed by addition of 20% Pip/DMF to execute deprotection in 5 min and 25 min sequentially. The resin was then washed by DMF/DCM, and drained. The automated CS336 Synthesizer was used to accomplish peptide synthesis till Met-1. Small scale cleavage showed the right mass. Carboxyfluorescein was attached to the resin using DIC/HOBt as coupling reagent. The resin was then washed with DMF/MeOH, drained, and ready for cleavage after vacuum drying. The dry peptidyl resin (0.5 g) was weighed and transferred to the reaction vessel. TFA solution containing appropriate scavengers was added. After 4 hours of reaction, the resin was removed by filtration under pressure and washed twice with TFA. The filtrates were combined. The filtrate volume was reduced by rotary evaporator, and cold ether was added to the residue to precipitate the crude peptide. The precipitated peptide was filtered through fritted funnel under a light vacuum, and further washed with cold ether for another 3 times. The crude peptide was then dried by air as an off-white powder, ˜190 mg. The peptide was dissolved in 0.1% TFA in water and ACN (Gradient: 50-70% ACN in 60 min, Flow rate: 28 mL/min), and fractions (peptide purity >95%) containing the expected MW were collected. The desired fractions were combined in a 1-liter lyophilizing jar and deeply frozen in liquid Nitrogen. The jar was later attached to VirTis lyophilizer for overnight drying under vacuum (<500 mTorr) to give the final peptide with TFA salt. The peptide was checked by analytical HPLC (Agilent 1200) using TFA buffer system, 1.5% per min gradient to give the purity >=99% QC. (ES)+-LCMS m/e found M.W. 3212.84 (expected 3212.66).

### Example 2

### Synthesis of Fluo-cyclo(Lys-Ile-Ile-Ile-Ile-Asp)-Gly-Ser-Thr-Ser-Arg-Asp-His-Nle-Val-Leu-His-Glu-Tyr-Val-Asn-Ala-Ala-Gly-Ile-Thr-Ado-NH₂ (SEQ ID NO: 3)

The peptide in this Example was prepared according to the method described in Example 1. (ES)+-LCMS m/e found M.W. 3320.64 (expected 3320.70).

### Example 3

### Synthesis of Fluo-cyclo(Lys-Nle-Ile-Ile-Leu-Ile-Ile-Asp)-Gly-Ser-Thr-Ser-Arg-Asp-His-Nle-Val-Leu-His-Glu-Tyr-Val-Asn-Ala-Ala-Gly-Ile-Thr-Ado-NH₂ (SEQ ID NO: 4)

The peptide in this Example was prepared according to the method described in Example 1. (ES)+-LCMS m/e found M.W. 3546.16 (expected 3547.02).

### Example 4

### Synthesis of Fluo-Nle-Ile-Ile-Leu-Ile-Ile-Gly-Ser-Thr-Ser-Arg-Asp-His-Nle-Val-Leu-His-Glu-Tyr-Val-Asn-Ala-Ala-Gly-Ile-Thr-Ado-NH₂ (SEQ ID NO: 5)

The peptide in this Example was prepared according to the method described in Example 1. (ES)+-LCMS m/e found M.W. 3320.56 (expected 3321.76).

### Example 5

### Synthesis of Fluo-(D-Met)-Ile-(D-Ile)-Leu-Ile-Ile-Gly-Ser-Thr-Ser-Arg-Asp-His-Nle-Val-Leu-His-Glu-Tyr-Val-Asn-Ala-Ala-Gly-Ile-Thr-Ado-NH₂ (SEQ ID NO: 6)

The peptide in this Example was prepared according to the method described in Example 1. (ES)+-LCMS m/e found M.W. 3339.32 (expected 3339.80).

### Example 6

### Synthesis of Fluo-Met-Ile-Ile-Leu-Ile-Ile-Met-Gly-Val-Ala-Asp-Leu-Ile-Lys-Lys-Phe-Glu-Ser-Ile-Ser-Lys-Glu-Glu-NH₂ (SEQ ID NO: 7)

The peptide in this Example was prepared according to the method described in Example 1. (ES)+-LCMS m/e found M.W. 2978.02 (expected 2978.52).

### Example 7

### Synthesis Fluo-Phe-Ile-cyclo(Asp-Ile-Ile-Ile-Lys)-Ile-Leu-Leu-Ile-Gly-Ser-Thr-Ser-Arg-Asp-His-Nle-Val-Leu-His-Glu-Tyr-Val-Asn-Ala-Ala-Gly-Ile-Thr-Ado-NH₂ (SEQ ID NO: 8)

The peptide in this Example was prepared according to the method described in Example 1. (ES)+-LCMS m/e found M.W. 3920.76 (expected 3920.51).

### Example 8

### Cellular Assays

The peptides of the invention were tested for cell penetration in HeLa and HEK293T cell lines.

Materials: The HeLa (DSMZ) and HEK293T cells were maintained in growth media and then passaged every 2-3 days. Growth media for HeLa cells was RPMI 1640, 10% fetal calf serum, MEM-non-essential amino acids, sodium pyruvate and L-glutamine (GIBCO). Growth media for HEK293T DMEM was supplemented with 10% fetal calf serum, MEM-non-essential amino acids, sodium pyruvate and glutamine (all GIBCO).

Methods and Procedures: Cells were plated onto μ-slide 8 well chambered coverslips (IBIDI) with ibidi standard-bottom and cultured overnight. Peptide stocks were prepared in MillQ and were diluted in cell growth media for cellular uptake studies. Cells were carefully washed with culture medium without fetal calf serum, incubated with 2 μM CPP concentration in absence of fetal calf serum and analyzed by confocal microscopy regarding uptake efficiency and localization over 2 h at 37° C. Finally, trypan blue (0.4% end concentration) was administered to quench cell surface fluorescence followed by an endpoint imaging step. Plates were imaged with an excitation at 488 nm and detection of fluorescence over 500-550 nm (fluorescein) using a TCS SP5 confocal microscope (Leica 174 Microsystems, Mannheim, Germany) equipped with an HCX PL APO 175 63×N.A. 1.2 water immersion lens and a temperature-controlled microscope stage.

The results for the peptides in Examples 1-7 in HeLa and HEK293T cells are shown in Table 1:

TABLE 1

| Cells | Ex. 1 | Ex. 2 | Ex. 3 | Ex. 4 | Ex. 5 | Ex. 6 | Ex. 7 |
|---|---|---|---|---|---|---|---|
| Jurkat (t2 h) | — | 0.9 | 0.77 | 0.85 | 0.18 | 0.44 | 1.35 |
| HEK293T (t2 h) | — | 0.23 | 0.35 | 0.16 | 0.5 | 0.08 | 0.28 |
| HeLa (t30 min) | 0.13 | — | — | — | — | — | — |
| HeLa(t2 h) | 0.11 | 0.18 | 0.28 | 0.12 | 0.22 | 0.14 | 0.28 |

As shown in the table above, penetration of HeLa cells was high for the peptide of Example 1 and fluorescence was localized in endosomes and cytosol. Penetration with the peptide of Example 7 was high and localized in endosomes followed by the peptide of Example 4, whereas low intracellular signals were measured in cells treated with the peptides of Examples 2, 3, 5 and 6. HEK293T cells showed pronounced cytosolic and partly endosomal fluorescence for the peptide of Example 7.

It is to be understood that the invention is not limited to the particular embodiments of the invention described above, as variations of the particular embodiments may be made and still fall within the scope of the appended claims.

1. A peptide, comprising the sequence of SEQ ID No. 1:
X_{A}-X_{B}-X_{C}-X_{D}-X_{E}-X_{F}-X_{G}-X_{H} (SEQ ID No. 1),
wherein:
X_{A} is absent or is Lys or Phe-Ile;
X_{B} is Met, Norleucine, Lys or Asp;
X_{C}, X_{D}, X_{E}, X_{F} and X_{G} are, independently of each other, a hydrophobic amino acid, Asp or Lys; and
X_{H} is absent or is Met, Asp or Leu-Leu-Ile,
said peptide optionally comprising a cargo moiety linked to a position on said sequence of SEQ ID No. 1 to form a peptide-cargo conjugate.

2. The peptide according to claim 1, wherein said hydrophobic amino acid is selected from the group consisting of Leu, Ile, Phe, Trp, Val, Met, Cys, Tyr and Ala.

3. The peptide according to claim 1, wherein said cargo moiety is a peptide, polypeptide, protein, small molecular substance, drug, mononucleotide, oligonucleotide, polynucleotide, antisense molecule, double stranded as well as single stranded DNA, RNA, an artificial or partly artificial nucleic acid, a low molecular weight molecule, saccharide, plasmid, antibiotic substance, cytotoxic agent, antiviral agent or a tag or marker molecule.

4. The peptide according to claim 1, wherein:
X_{A} is absent;
X_{B} is Met or Norleucine;
X_{C}, X_{D} and X_{E} independently are a hydrophobic amino acid;
X_{F} and X_{G} independently are a hydrophobic amino acid;
X_{H} is absent or is Met;
and wherein X_{H} or, in the absence of X_{H}, X_{G} is optionally linked to said cargo moiety.

5. The peptide according to claim 1, wherein:
X_{A} is absent;
X_{B} is Met or Norleucine;
X_{C}, X_{D} and X_{E} independently are Ile or Leu;
X_{F} and X_{G} independently are a hydrophobic amino acid;
X_{H} is absent or is Met;
and wherein X_{H} or, in the absence of X_{H}, X_{G} is optionally linked to said cargo moiety.

6. The peptide according to claim 1, wherein:
X_{A} is absent or is Lys or Phe-Ile;
X_{B} is Norleucine, Lys or Asp;
X_{C}, X_{D} and X_{E} independently are a hydrophobic amino acid;
X_{F} and X_{G} independently are a hydrophobic amino acid;
X_{H} is absent or is Met, Asp or Leu-Leu-Ile;
X_{A} or X_{B} and X_{F}, X_{G} or X_{H} are optionally linked to form a lactam bridge;
and wherein X_{H} or, in the absence of X_{H}, X_{G} is optionally linked to said cargo moiety.

7. The peptide according to claim 1, wherein:
X_{A} is absent or is Lys or Phe-Ile;
X_{B} is Norleucine, Lys or Asp;
X_{C}, X_{D} and X_{E} independently are Ile or Leu;
X_{F} and X_{G} independently are Ile, Asp or Lys;
X_{H} is absent or is Met, Asp or Leu-Leu-Ile;
X_{A} or X_{B} and X_{F}, X_{G} or X_{H} are optionally linked to form a lactam bridge;
and wherein X_{H} or, in the absence of X_{H}, X_{G} is optionally linked to said cargo moiety.

8. A peptide-cargo conjugate, comprising:
a peptide comprising the sequence of SEQ ID No. 1:
X_{A}-X_{B}-X_{C}-X_{D}-X_{E}-X_{F}-X_{G}-X_{H} (SEQ ID No. 1),
wherein:
X_{A} is absent or is Lys or Phe-Ile;
X_{B} is Met, Norleucine, Lys or Asp;
X_{C}, X_{D}, X_{E}, X_{F} and X_{G} are, independently of each other, a hydrophobic amino acid, Asp or Lys; and
X_{H} is absent or is Met, Asp or Leu-Leu-Ile; and
a cargo moiety linked to a position on said sequence of SEQ ID No. 1.

9. A pharmaceutical composition, comprising a therapeutically effective amount of a peptide-cargo conjugate according to claim 1.

10. A method for the treatment of a cancerous, infectious, neurological, inflammatory, immunological, ocular or metabolic disease or disorder, comprising the step of administering a therapeutically effective amount of a peptide-cargo conjugate according to claim 1 to a patient in need thereof.

11-13. (canceled)
